(19) United States
(12) Patent Application Publication
(10) Pub. No.: US 2026/0216189 A1
(43) Pub. Date: Jul. 30, 2026

(54) TOPICAL COMPOSITIONS CONTAINING A JANUS KINASE INHIBITOR

(71) Applicant: Zoetis Services LLC, Parsippany, NJ (US)

(72) Inventors: Debendra Kumar Panda, Navi Mumbai (IN); Kritika Nayak, Navi Mumbai (IN)

(21) Appl. No.: 19/158,210

(22) PCT Filed: Mar. 1, 2024
(86) PCT No.: PCT/US2024/018012

§ 371 Date: Aug. 20, 2025

Related U.S. Application Data
(60) Provisional application No. 63/488,544, filed on Mar. 6, 2023.

(51) Int. Cl. A61K 31/519; A61K 9/00; A61K 9/08; A61K 31/4439; A61K 47/02; A61K 47/10; A61K 47/14; A61K 47/20

(52) U.S. Cl. CPC A61K 31/519; A61K 9/0017; A61K 9/08; A61K 31/4439; A61K 47/02; A61K 47/10; A61K 47/14; A61K 47/20

(57) ABSTRACT

The present invention describes a topical veterinary solution composition comprising a JAK inhibitor, preferably oclacitinib, or a pharmaceutically acceptable salt thereof, for use on animals, preferably canine, for the treatment of dermatological disorders, for example, allergic dermatitis, atopic dermatitis, pruritis and/or a hot spot(s).

### FIELD OF THE INVENTION

Described herein are topical compositions comprising a Janus Kinase (JAK) inhibitor (JAKi); and in particular, N-methyl-1-{trans-4-[methyl(7H-pyrrolo[2,3-d]pyrimidin-4-yl)amino]cyclohexyl}-methanesulfonamide (oclacitinib) or a pharmaceutically acceptable salt (e.g., maleate) thereof.

### BACKGROUND OF THE INVENTION

Protein kinases are families of enzymes that catalyze the phosphorylation of specific residues in proteins, broadly classified into tyrosine and serine/threonine kinases. Inappropriate kinase activity, arising from mutation, over-expression, or inappropriate regulation, dis-regulation or de-regulation, as well as over- or under-production of growth factors or cytokines has been implicated in many diseases, including but not limited to cancer, allergies, asthma and other respiratory diseases, autoimmune diseases, and inflammatory diseases. Inappropriate kinase activity triggers a variety of biological cellular responses relating to cell growth, cell differentiation, survival, apoptosis, mitogenesis, cell cycle control, and cell mobility implicated in the aforementioned and related diseases.

Thus, protein kinases have emerged as an important class of enzymes as targets for therapeutic intervention. In particular, the JAK family of cellular protein tyrosine kinases (JAK-1, JAK-2, JAK-3, and Tyk-2) play a central role in cytokine signaling (Kisseleva et al, Gene, 2002, 285, 1; Yamaoka et al. Genome Biology 2004, 5, 253)). Upon binding to their receptors, cytokines activate JAK which then phosphorylate the cytokine receptor, thereby creating docking sites for signaling molecules, notably, members of the signal transducer and activator of transcription (STAT) family that ultimately lead to gene expression. Numerous cytokines are known to activate the JAK family.

Patent applications have recently published (e.g., WO2021/014453, WO2022/013708 and WO2022/027041) with topical compositions for other JAK inhibitors. WO2021/014453 describes emulsion based topical creams containing tofacitinib, retinoids, menthol, oils and alcohols. WO2022/013708 describes oil-in-water emulsions containing a JAK inhibitor, white petrolatum (10%) and an antimicrobial agent. WO2022/027041 describes a topical oil-in-water emulsion composition containing a JAK inhibitor (SHR0302), laureth-4 and a solvent. Accordingly, laureth-4, a synthetic polymer composed of lauryl alcohol and polyethylene glycol, was shown to enhance skin permeation of this particular JAK inhibitor. In addition, US Patent application US2020-0352965 describes a topical ointment with 70% white petrolatum with tofacitinin and crisaborole. US patent application US2020-0276109 describes an ointment composition containing the JAK inhibitor (3aR,5s,6aS)-N-(3-methoxy-1,2,4-thiadiazol-5-yl)-5-(methyl (7H-pyrrolo[2,3-d]pyrimidin-4-yl)amino)hexahydrocyclopenta[c]pyrrole-2 (1H)-carboxamide with diethylene glycol monoethyl ether (DEGMEE). Unlike these oil-in-emulsion, cream and ointment based compositions, the current invention describes a solution based composition that can be easily sprayed on the skin of an animal in need thereof.

Current dosage forms containing oclacitinib include orally administered film-coated tablets and flavored chewable tablets. In lieu of these oral doses, there is also a need to administer oclacitinib topically to control site specific areas (e.g., hot spots) of dermatitis. An earlier patent application publication, WO2006/069080, merely describes topical administration of a JAK inhibitor from a broad genus of JAK inhibitors; however, the citation does not describe any particular compound or composition that could actually be prepared and/or administered topically. As such, a topical spray solution composition was developed for use on animals, particularly canines.

### SUMMARY OF THE INVENTION

The present invention provides a topical solution spray composition comprising a JAK inhibitor. In one aspect, the JAK inhibitor (JAKi) is a 7H-pyrrolo[2,3-d]pyrimidine compound of Formula A, that is

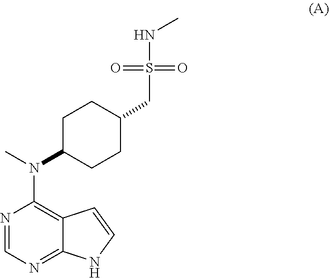

N-methyl-1-{trans-4-[methyl (7H-pyrrolo[2,3-d]pyrimidin-4-yl)amino]cyclohexyl}-methanesulfonamide (oclacitinib), as a free base, or a veterinary acceptable salt thereof. A preferred salt form of Formula A is the maleate salt.

In another aspect, is a topical composition comprising a JAKi compound of Formula (B), Formula (C) or Formula (D), either as a free base or a pharmaceutically acceptable salt thereof.

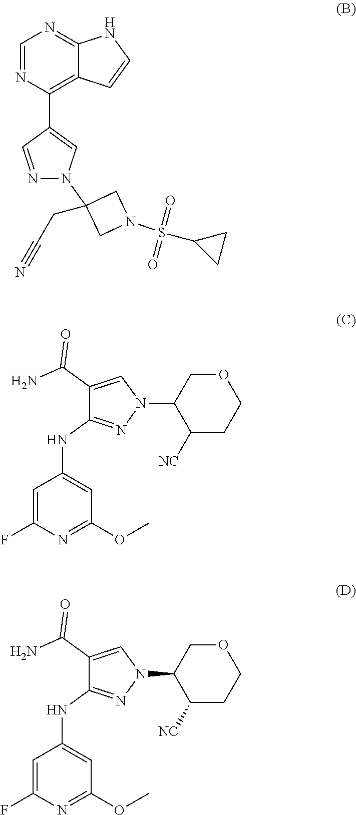 [SMILES: N#CCC1(n2cc(-c3ncnc4[nH]ccc34)cn2)CN(S(=O)(=O)C2CC2)C1 ; COc1cc(Nc2nn(C3COCCC3C#N)cc2C(N)=O)cc(F)n1 ; COc1cc(Nc2nn([C@H]3COCC[C@@H]3C#N)cc2C(N)=O)cc(F)n1]

In another aspect, the topical composition comprises a JAKi compound of Formula (A), Formula (B), Formula (C) or Formula (D), or a pharmaceutically acceptable salt thereof, in combination with at least one pharmaceutically acceptable excipient. The amount of the API, as described herein, is the weight of the freebase of the agent, i.e., not including the total weight of the compound when associated with a pharmaceutically acceptable salt. For example, 10 mg/ml of the Formula (A) compound is based on the freebase amount of the compound. In another aspect, the topical composition comprises the JAKi compound of Formula (A), or a pharmaceutically acceptable salt thereof, with at least one pharmaceutically acceptable excipient. In another aspect, the topical composition comprises the JAKi compound of Formula (B), or a pharmaceutically acceptable salt thereof, with at least one pharmaceutically acceptable excipient. In another aspect, the topical composition comprises the JAKi compound of Formula (C), or a pharmaceutically acceptable salt thereof, with at least one pharmaceutically acceptable excipient. In another aspect, the topical composition comprises the JAKi compound of Formula (D), or a pharmaceutically acceptable salt thereof, with at least one pharmaceutically acceptable excipient. In one aspect, the pharmaceutically acceptable excipient comprises a solvent or mixture of solvents. In another aspect, the pharmaceutically acceptable excipient comprises a solvent or mixture of solvents and optionally, a surfactant, antioxidant and pH modifier. In another aspect, the composition comprises a mixture of solvents selected from a glycol, glycol ether, alcohol, dimethylsulfoxide and water; and optionally, a surfactant, antioxidant and pH modifier. In another aspect, the composition comprises a mixture of solvents selected from a glycol, glycol ether, dimethylsulfoxide and water; and optionally, a surfactant, antioxidant and pH modifier. In another aspect, the composition comprises a mixture of solvents selected from propylene glycol, a glycol ether selected from the group consisting of dipropylene glycol monomethyl ether (DPGMME), diethylene glycol monoethyl ether (DEGMEE) and diethylene glycol monobutyl ether (DEGMBE); dimethylsulfoxide, an alcohol, water; and optionally, a surfactant, antioxidant and pH modifier. In another aspect, the composition comprises a mixture of solvents selected from propylene glycol, a glycol ether selected from DPGMME, DEGMEE or DEGMBE; dimethylsulfoxide, water; and optionally, a surfactant, antioxidant and pH modifier. In another aspect, the solvent mixture comprises propylene glycol, a glycol ether selected from DPGMME, DEGMEE or DEGMBE; dimethylsulfoxide, ethanol and water; and optionally a surfactant, antioxidant and pH modifier. In another aspect, the solvent mixture comprises propylene glycol, a glycol ether selected from DPGMME, DEGMEE or DEGMBE; dimethylsulfoxide and water; and optionally a surfactant, antioxidant and pH modifier. In another aspect, the solvent mixture comprises propylene glycol, DEGMEE, dimethysulfoxide, ethanol and water; and optionally, a surfactant, antioxidant and pH modifier. In another aspect, the solvent mixture comprises propylene glycol, DEGMEE, dimethysulfoxide and water; and optionally, a surfactant, antioxidant and pH modifier. In another aspect, the composition comprises propylene glycol, DEGMEE, dimethylsulfoxide, ethanol, water, a surfactant; and optionally, an antioxidant and pH modifier. In another aspect, the composition comprises propylene glycol, DEGMEE, dimethylsulfoxide, water, a surfactant; and optionally, an antioxidant and pH modifier. In another aspect, the composition comprises propylene glycol, DEGMEE, dimethylsulfoxide, ethanol, water, a surfactant and an antioxidant, and optionally, a pH modifier. In another aspect, the composition comprises propylene glycol, DEGMEE, dimethylsulfoxide, water, a surfactant and an antioxidant, and optionally, a pH modifier. In another aspect, the composition comprises propylene glycol, DEGMEE, dimethylsulfoxide, ethanol, water, a surfactant, an antioxidant and pH modifier. In another aspect, the composition comprises propylene glycol, DEGMEE, dimethylsulfoxide, water, a surfactant, an antioxidant and pH modifier. In another aspect, the composition comprises propylene glycol, DEGMEE, dimethylsulfoxide, ethanol, water, an antioxidant and pH modifier. In another aspect, the composition comprises propylene glycol, DEGMEE, dimethylsulfoxide, water, an antioxidant and pH modifier. In another aspect, the composition comprises propylene glycol, DEGMEE, dimethylsulfoxide, ethanol, water and an antioxidant. In another aspect, the composition comprises propylene glycol, DEGMEE, dimethylsulfoxide, water and an antioxidant. In another aspect, the antioxidant is BHT or BHA and the pH modifier is sodium hydroxide. In another aspect, the composition comprises about 1-15 mg/ml of a Formula (A), Formula (B), Formula (C) or Formula (D) compound, or pharmaceutically acceptable salt thereof; about 0-15 w/v % of a glycol; about 10-40 w/v % of a glycol ether; about 0-15 w/v % of an alcohol; about 25-50 w/v % of DMSO; and water; and optionally, a surfactant, antioxidant and pH modifier. In another aspect, the composition comprises about 1 mg/mL to about 10 mg/mL of a Formula (A) compound, or pharmaceutically acceptable salt thereof; about 0 w/v % to about 15 w/v % of a glycol; about 10 w/v % to about 40 w/v % of a glycol ether; about 0 w/v % to about 15 w/v % of an alcohol; about 25 w/v % to about 50 w/v % of DMSO; and water; and optionally, a surfactant, antioxidant and pH modifier. In another aspect, the composition comprises about 1 mg/mL to about 10 mg/ml of a Formula (A) compound, or pharmaceutically acceptable salt thereof; about 1 w.v % to about 10 w/v % of a glycol; about 20 v/v % to about 35 w/v % of a glycol ether; about 0 w/v % to about 10 w/v % of an alcohol; about 30 w/v % to about 50 w/v % of DMSO; and water; and optionally, a surfactant, antioxidant and pH modifier. In another aspect, the composition comprises about 1 mg/mL to about 10 mg/ml of a Formula (A) compound, or pharmaceutically acceptable salt thereof; about 1 w/v % to about 10 w/v % of propylene glycol; about 20 w/v % to about 35 w/v % of a glycol ether selected from DPGMME, DEGMEE and DEGMBE; about 0 w/v % to about w/v % of an alcohol; about 30 w/v % to about 45 w/v % of DMSO; and water; and optionally, a surfactant, antioxidant and pH modifier. In another aspect, the composition comprises about 1 mg/mL to about 10 mg/ml of a Formula (A) compound, or pharmaceutically acceptable salt thereof; about 2 w/v % to about 8 w/v % of propylene glycol; about 25 w/v % to about 35 w/v % of a glycol ether selected from DPGMME, DEGMEE and DEGMBE; about 0 w/v % to about 10 w/v % of ethanol; about 30 w/v % to about 45 w/v % of DMSO; and water; and optionally, about 0 w/v % to about 6 w/v % of Labrasol®, and the antioxidant BHA or BHT and a pH modifier. In another aspect, the composition comprises about 1 mg/mL to about 10 mg/ml of a Formula (A) compound, or pharmaceutically acceptable salt thereof; about 2 w/v % to about 8 w/v % of propylene glycol; about 25 w/v % to about 35 w/v % of DEGMEE; about 0 w/v % to about 10 w/v % of ethanol; about 30 w/v % to about 45 w/v % of DMSO; and water; and optionally, BHA or BHT and a pH modifier. In another aspect, the composition comprises about 1 mg/ml to about 10 mg/ml of a Formula (A) compound, or pharmaceutically acceptable salt thereof; about 2 w/v % to about 8 w/v % of propylene glycol; about 25 w/v % to about 35 w/v % of DEGMEE; about 0 w/v % to about 10 w/v % of ethanol; about 30 w/v % to about 45 w/v % of DMSO; water; and BHA or BHT; and optionally, a pH modifier. In another aspect, the composition comprises about 1 mg/mL to about 10 mg/ml of a Formula (A) compound, or pharmaceutically acceptable salt thereof; about 2 w/v % to about 8 w/v % of propylene glycol; about 25 w/v % to about 35 w/v % of DEGMEE; about 0 w/v % to about 10 w/v % of ethanol; about 30 w/v % to about 45 w/v % of DMSO; water; BHA or BHT; and a pH modifier. In all aspects of the invention; about 0 w/v % can be zero w/v % (i.e. not included in the composition). In another aspect, the composition comprises about 1 mg/mL to about 5 mg/ml of a Formula (A) compound, or pharmaceutically acceptable salt thereof; about 2 w/v % to about 8 w/v % of propylene glycol; about 25 w/v % to about 35 w/v % of DEGMEE; about 30 w/v % to about 45 w/v % of DMSO; water; BHA or BHT; and a pH modifier that is sodium hydroxide and wherein the pH ranges from about 4.8 to 6.0. In another aspect, the composition comprises about 1 mg/mL to about 5 mg/mL of a Formula (A) compound, or pharmaceutically acceptable salt thereof; about 2 w/v % to about 8 w/v % of propylene glycol; about 25 w/v % to about 35 w/v % of DEGMEE; about 40 w/v % DMSO; water; BHA or BHT; and a pH modifier that is sodium hydroxide and wherein the pH ranges from about 4.8 to 6.0. In another aspect, the composition comprises about 1 mg/mL to about 5 mg/ml of a Formula (A) compound, or pharmaceutically acceptable salt thereof; about 5 w/v % of propylene glycol; about 30 w/v % of DEGMEE; about 40 w/v % of DMSO; water; BHA or BHT; and a pH modifier that is sodium hydroxide and wherein the pH ranges from about 4.8 to 6.0. In another aspect, the composition comprises about 1 mg/mL to about 5 mg/ml of a Formula (A) compound, or pharmaceutically acceptable salt thereof; about 5 w/v % of propylene glycol; about 30 w/v % of DEGMEE; about 35 w/v % of DMSO; about 1 w/% to about 5 w/v % of ethanol, water; BHA or BHT; and a pH modifier that is sodium hydroxide and wherein the pH ranges from about 4.8 to 6.0. In another aspect, the composition comprises about 1 mg/mL to about 5 mg/mL of a Formula (A) compound, or pharmaceutically acceptable salt thereof; about 5 w/v % of propylene glycol; about 30 w/v % of DEGMEE; about 40 w/v % of DMSO; about 1 w/% to about 5 w/v % of ethanol, water; BHA or BHT; and a pH modifier that is sodium hydroxide and wherein the pH ranges from about 4.8 to 6.0. In another aspect, the composition of the invention is administered topically to an animal in need thereof, to treat or prevent a dermatological disease or disorder. In another aspect, the dermatological disease or disorder is atopic dermatitis, allergic dermatitis, pruritis or a hot spot. In another aspect, the composition of the invention is administered topically to an animal in need thereof, to treat or prevent atopic dermatitis, allergic dermatitis or a hot spot. In another aspect, the composition of the invention is administered topically to an animal in need thereof, to treat or prevent atopic dermatitis or allergic dermatitis. In another aspect, the composition of the invention is administered topically to an animal in need thereof, to treat or prevent atopic dermatitis. In another aspect, the composition of the invention is administered topically to an animal in need thereof, to treat or prevent allergic dermatitis. In another aspect, the composition of the invention is administered topically to an animal in need thereof, to treat a hot spot. In another aspect, the animal is equine, canine or feline. In another aspect, the animal is canine.

In another aspect, the composition of the invention is used to prepare a medicament to be administered topically to an animal in need thereof, to treat or prevent atopic dermatitis, allergic dermatitis or a hot spot.

In another aspect, the composition of the invention can be co-administered with another pharmaceutical agent that is administered orally, topically or by injection; and/or with cleansing agent(s). Non-exclusive examples of other pharmaceutical agents include nutritional supplements, antiparasitics, antibacterials, antifungals, anti-inflammatories and dermatologicals (e.g., JAK inhibitors, cyclosporin, prednisone, dexamethasone, betamethasone, and the like). Non-limiting examples of cleansing agents include for example: hydrogen peroxide, medicated shampoo, chlorhexidine gluconate, benzoyl peroxide, and the like.

### DESCRIPTION

### Definitions

For purposes of the present invention, as described and claimed herein, the following terms and phrases are defined as follows:

“About” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater. In some jurisdictions, the term is considered ambiguous, as such, the term can be readily deleted from the claims thereby ensuring the range as claimed.

“Active pharmaceutical ingredient” (API) or “active agent”, as used herein, unless indicated otherwise, are interchangeable and mean the ingredient is a pharmaceutical drug which is biologically active, for example, a JAK inhibitor.

“Animal” as used herein, unless otherwise indicated, refers to an individual animal, and said individual animal is a mammal. Specifically, mammal refers to a vertebrate animal that is human and non-human, which are members of the taxonomic class Mammalia. Non-exclusive examples of non-human animals include companion animals and livestock. Non-exclusive examples of a companion animal include: dog (canine), cat (feline) and horse (equine). A preferred companion animal is a dog. Non-exclusive examples of livestock include: pigs (porcine), rabbit (leporidae), goat (caprine), sheep (ovine) and cattle (bovine).

“Clinical sign” as used herein, unless otherwise indicated, refers to an observable or measurable condition or behavior in the animal that is indicative of the disease, condition or symptom. Clinical signs may be those symptoms, conditions, or behaviors that are measured in known or established diagnostic assessments. For example, diagnostic assessments for a determination of allergic dermatitis or atopic dermatitis can be made by a Visual Analog Scale (VAS) Score or a clinical assessment of condition, or by an established scoring system such as the Canine Atopic Dermatitis Extent and Severity Index (CADESI) Score. Non-limiting examples of some clinical signs for atopic dermatitis, allergic dermatitis or a hot spot(s), that may be used sometimes in such assessments or scoring systems, include: itching, ranging from extremely severe (as demonstrated, in the case of a companion animal such as a dog, by scratching, chewing, licking almost continuously, regardless of what else is happening), to severe (as demonstrated by prolonged episodes of itching while awake, and itching at night and/or while eating, playing or exercising), to moderate (as demonstrated by frequent episodes of itching), to very mild (occasional episodes of itching); presence of pustules or epidermal collarets; presence of skin lesions; pruritus; erythema; erosions, excoriations and/or self-induced alopecia; presence of papules and/or crusts; lichenification and/or hyperpigmentation.

“Composition(s) of the invention”, as used herein, unless otherwise indicated, refers to a composition that is a solution, comprising a JAK inhibitor of Formula (A), Formula (B), Formula (C) or Formula (D), or a pharmaceutically acceptable salt thereof; preferably Formula (A), and at least one pharmaceutically acceptable excipient selected from the group of a glycol, glycol ether, DMSO and water, and optionally further comprising an alcohol, surfactant, antioxidant and pH modifier.

“Hot spot” as used herein, unless otherwise indicated, refers to an inflammatory skin lesion or skin irritation on an animal that can be exacerbated by scratching and licking and is also know as pyotraumatic dermatitis or acute moist dermatitis. Hot spots are red, inflamed skin lesions.

“Pharmaceutically acceptable” as used herein, unless otherwise indicated, suggests that the substance or composition must be compatible chemically and/or toxicologically with the other ingredients comprising the composition and/or the animal being treated therewith. The term “pharmaceutically” encompasses “veterinarily acceptable”.

“Pharmaceutically acceptable salt” as used herein, unless otherwise indicated, refers to any salt suitable for pharmaceutical use in an animal, preferably the acetate, ascorbate, aspartate, benzoate, besylate, bicarbonate/carbonate, bisulphate/sulphate, borate, camsylate, citrate, edisylate, etoglutarate, esylate, formate, fumarate, gluceptate, gluconate, glucuronate, glycerophosphate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate or trifluoroacetate salt. Preferably the pharmaceutically acceptable salt for oclacitinib is the maleate (or maleic acid) salt.

“Quantum satis” or “qs”, as used herein, unless otherwise indicated, refers to the amount that is as much as is sufficient; or in other words, the amount of solvent (e.g., water) required to bring the final w/v % of the solution to 100%.

“Therapeutically effective amount”, as used herein, unless otherwise indicated, refers to an amount of a JAK inhibitor, preferably oclacitinib (free base), to treat or prevent a disease, condition or disorder associated with JAK in an animal, i.e., a disease or condition caused by or associated with an immune system dysfunction or immune system dysregulation; comprising topically administering an effective amount of a JAK inhibitor described herein to the animal.

“Treating” or “treatment” as used herein, unless indicated otherwise, means controlling, treating, or preventing the progression of the indicated condition or disease. The term “controlling”, “treating” or “treatment” of a condition or disease includes: (1) preventing the condition or disease, i.e. causing the clinical symptoms or signs of the disease not to develop in a mammal that may be exposed to or predisposed to the disease but does not yet experience or display symptoms/signs of the disease; (2) inhibiting the disease, i.e., arresting or reducing the progression of the disease or its clinical symptoms or signs; or (3) relieving the disease, i.e., causing regression of the disease or its clinical symptoms or signs.

A “symptom” of a disease or condition is any of those symptoms known by a person of ordinary skill in the art as being associated with the disease or condition. In the case of atopic dermatitis, allergic dermatitis, flea allergy dermatitis, and sarcoptic mange, symptoms include, for example: pruritus, itch, and skin lesions. In many cases, a “symptom” of a disease or condition, such as atopic dermatitis or allergic dermatitis is also a “clinical sign”. In the case of allergic dermatitis, the allergic dermatitis may be flea allergy dermatitis, i.e. “FAD” (also called “flea allergic dermatitis”, “flea bite dermatitis” (“FBD”), or “flea-associated dermatitis), food allergy dermatitis, contact dermatitis, or allergic dermatitis associated with Sarcoptes scabiei (i.e. sarcoptic mange). Other indications and conditions that can be treated by the methods including the dosing regimens described herein include any indications or conditions treatable by administration of a JAK inhibitor, including those involving JAK-1, JAK-2 or JAK-3. Such indications and conditions include cancer, asthma, atopic dermatitis, allergic dermatitis, autoimmune thyroid disorders, pruritus, chronic respiratory disease and other indications where immunosuppression or immunomodulation would be desirable.

As used herein, percent of components of the composition refers to weight/volume percentages of the total weight of the composition and is referred to as “w/v %” which defines the mass fraction of the compositional component expressed as a percentage, determined according to the formula mᵢ/mₜₒₜ×100, wherein mᵢ is the mass of the substance of interest present in the composition, and mₜₒₜ is the total mass of the composition.

The pharmacokinetics and pharmacodynamics (PK/PD) of orally dosed oclacitinib (0.4-0.6 mg/kg) is well understood. Plasma concentrations of greater than 12 ng/ml have been shown to be effective in reducing IL-31 induced itch. An oral 14C-oclacitinib study determined that a single 0.4 mg/kg dose achieved a mean skin concentration of 212 ng equivalents/gram of tissue. A house dust model (HDM) was used to assess the efficacy of the compositions described herein. The topical HDM challenge results in excoriation lesions and erythema at the challenge site. In one study, placebo (saline, TO1), triamcinolone acetonide 0.15 mg/mL (T02), oclacitinib maleate 7.5 mg/mL (T03) and oclacitinib maleate 10 mg/mL (T04) were topically administered to beagle dogs (8/group). The weights of drug in T02-T04 were freebase amounts. Animals received about 1 mL of each of the solutions according to the following treatment/dosing regimen as shown in Table 1.

TABLE 1
Treatment Regimens

| | | | Target |
|---|---|---|---|
| | Dose | | Daily |
| | Concentration | | Dose |
| Treatment | (mg/mL) | Topical Regimen | (mg/kg) |
| T01 | 0 | 6 sprays (~1 mL) once daily for | 0 |
| | | 28 days | |
| T02 | 0.15 | 2 sprays (~1 mL) twice daily for | 0.03 and |
| | | 7 days, once daily for 7 days then | 0.015^{b} |
| | | every other day for 14 days^{a} | |
| T03 | 7.5 | 6 sprays (~1 mL) once daily for | 0.75 |
| | | 28 days | |
| T04 | 10 | 6 sprays (~1 mL) once daily for | 1.0 |
| | | 28 days | |

^{a}Commercial dosing regimen
^{b}0.03 mg/kg daily dose for 7 days then 0.015 mg/kg daily dose

Daily assessments were observed through Day 41. Colorimeter measurements provided quantified erythema scores. Both concentrations of oclacitinib achieved significant erythema reductions compared to saline after approximately 1 month of dosing. The triamcinolone spray had significantly less reduction in erythema compared to saline as early as Day 12, coinciding with a tapering of the dosing frequency, and continuing for multiple timepoint comparisons throughout the observation period. Triamcinolone treated animals had significantly less reduction in erythema compared to those treated with saline on Days 12, 14, 19, 24-26, 28-33, and 35-41 (P≤0.0912). Animals dosed with oclacitinib at 7.5 mg/mL had significantly greater reduction in erythema compared to those dosed with saline on Day 36 (P=0.0993). Animals dosed with oclacitinib at 10 mg/mL had significantly greater reduction than saline dosed animals on Days 27, 28, and 36 (p≤0.0948). Lesions were observed and scored based on severity of erythema, papules and excoriations at the challenge site. Post challenge total lesion scores were significantly higher for the triamcinolone and oclacitinib treatment groups compared to the saline group. Triamcinolone treated animals had significantly greater reductions in lesion scores compared to saline treated animals on Days 4, 8, 10, 11, but a significantly lesser reduction on Day 35. Animals treated with oclacitinib spray at 7.5 mg/mL had significantly greater lesion score reductions compared to animals treated with saline on Days 4, 7, 11, 26, 27, 30, and 39. Animals treated with oclacitinib spray at 10 mg/mL had significantly greater lesion score reductions than saline treated animals on Days 7-11, 26, 30, and 32-34. Oclacitinib treatment groups continued to demonstrate significant reduction in lesion scores after the dosing period ended than the other treatment groups. Animals treated with 10 mg/mL oclacitinib had a significantly lower area under the curve (AUC) of total lesion scores compared to the animals treated with saline (p<0.08). For similar AUC lesion scores, no other treatments tested were significantly different than the saline control.

In a subsequent study, animals received 2.5-10 mg/mL oclacitinib by applying 2-3 sprays for a total of about 0.25 to 0.5 mL of the topical solution to an area of skin. This study also provided acceptable skin concentrations of oclacitinib.

Topical administration includes the treatment of skin readily accessible by local application to generate a local and/or systemic effect. The term “topically” is used herein in its conventional sense to refer to the route of administration where the active is delivered across a portion of the surface of the skin, such as delivered to one or more of the subcutis, dermis and epidermis, including the stratum corneum, stratum germinativum, stratum spinosum and stratum basale. Accordingly, topical compositions having an API are formulated to be applied to any location on the animal. In some aspects, the skin is healthy intact skin. In other aspects, the skin may be skin where one or more layers (e.g., stratum corneum, stratum germinativum, stratum spinosum, stratum basale, etc.) may be diseased or inflamed. In certain aspects, the API is delivered locally to the site of administration. The term “locally” is used herein in its conventional sense to mean that the API is delivered within the vicinity or underlying vicinity of the application site. Based on the size of the spray head, volumes administered per pump (stroke) can range from about 0.05 to about 0.3 mL; or from about 0.08 to about 0.25 mL; or about 0.1 to about 0.25 mL; or about 0.15 to about 0.25 mL; or about 0.1 to about 0.2 mL. Further, depending on the distance from the skin (e.g., 3, 4, 5, 6 inches) when pumped (e.g., 1×, 2×, 3× and the like), the composition can cover an area of skin ranging from about 10 cm² to about 125 cm². For example, a 0.2 mL volume spray can cover about 25 cm² at about 3-6 inches; or about 40 cm² at 4-7 inches; or about 55 cm² at 5-8 inches. Similarly, a 0.4 mL (2×) volume spray can cover about 50 cm² at about 4-6 inches. If the composition comprises a 5 mg/mL concentration of the API, then a single 0.2 mL pump will spray an amount of about 1 mg of API and if the area sprayed is about 25 cm², then the amount of API administered to the site will be about 1 mg/25 cm² or 0.04 mg/cm². Overall, drug concentration administered to the skin will vary depending on composition API concentration (mg/mL), pump volume (mL), number of pumps, distance from skin (inches) and area of skin (cm²) treated. In some aspects, the API diffuses from the application site by as much as 12 cm or less; 10 cm or less; 8 cm or less; 6 cm or less; 4 cm or less; 2 cm or less; or 1 cm or less from the application site. In other words, in these aspects, the topical compositions are administered with the intention of delivering the API to a location at or within a small distance from the site of administration. The penetration depth of the API administered locally into the skin at the site of application may also vary depending on the components of the composition. In some instances, topical administration of the API may also result in systemic absorption of the API.

Whenever a numerical range is indicated herein, it is meant to include any cited numeral (fractional or integral) within the indicated range. The terms comprises, comprising, includes, including means “including but not limited to”. As used herein, the indefinite articles “a” and “an” mean at least one or one or more unless the context clearly dictates otherwise.

Clinical signs of allergic and atopic dermatitis in the skin includes, for example, erythema (redness) and alopecia (hair loss). The skin lesions associated with allergic dermatitis and atopic dermatitis can vary significantly in a clinical setting. The most common location of localized skin lesions are the ears, abdomen, inguinal areas and/or feet. In addition, secondary superficial bacteria and/or yeast infections may be present, resulting in the development of a pyotraumatic dermatitis, colloquially known as a hot spot. Unlike oral Apoquel® (oclacitinib maleate), which acts systemically, the goal of topical therapy is to provide a treatment to a very specific area associated with atopic and/or allergic dermatitis and hot spot(s). Topical treatment will provide a higher skin concentration at the target area thereby leading to increased efficacy. Hence, the topical oclacitinib composition must penetrate the skin and achieve sufficient skin concentrations.

Systemic plasma concentrations and skin concentrations 24 hours post dose were assessed for multiple compositions of the invention in a number of PK studies; including compositions with oclacitinib or oclacitinib maleate with API concentrations ranging from 1-10 mg/mL oclacitinib. The compositions tested to date have shown prolonged absorption that lasts at least 72 hours following a single dose and low topical bioavailability of less than 10% with single dose skin concentrations at least 20-80 times that observed in the oral single dose ¹⁴C study. The oral 14C-oclacitinib study determined that a 0.4 mg/kg oral dose of oclacitinib achieved a mean skin concentration of 212 ng equivalents/gram. Permeation and penetration studies with compositions comprising a glycol, glycol ether and DMSO achieved single dose skin concentrations greater (28.4-54.5 μg/g) than that concentration achieved following oral administration, as mentioned above, and well above the reported oclacitinib IC₅₀'s for pro-inflammatory and pruritogenic cytokines.

Across study comparisons demonstrated that the 24-hour post dose skin concentrations were at least 5 times higher following repeat dosing. Systemic plasma concentrations in the single and repeat dose studies showed that there is prolonged absorption with the terminal phase not evident 10 days post dose. The rate and amount of topical absorption can be a non-linear function of the API concentration of the composition. The topical compositions of the invention minimized systemic plasma oclacitinib concentrations while maintaining high skin concentrations. The PK following topical administration of oclacitinib maleate and oclacitinib freebase formulations were comparable. The studies to date and formulations described herein have shown that the compositions deliver skin concentrations greater than those observed with oral dosing. The oclacitinib formulations with an oclacitinib concentration of 7.5 mg/ml exhibited prolonged absorption with a duration of greater than 7 days following a single dose. The topical single dose bioavailability is low, less than 5% when administered to an area that limits oral exposure from licking. However, low bioavailability does not equal low exposure. Accumulation in skin and plasma was observed following multiple daily administrations. The systemic levels following multiple daily doses were at a concentration that has shown efficacy in reducing IL-31 induced itch. Formulations with a lower oclacitinib concentration had lower systemic exposures.

The resulting composition of the invention is a solution. That can be administered as an aerosol (inclusion of a propellant (e.g., carbon dioxide, a hydrofluoroalkane, and the like)) or spray. The preferred topical composition is a solution that can be sprayed.

Depending on the site of application and physiology of the subject and surface area on the skin applied with the topical composition, the amount of the active agent in the composition may vary, in some instances, the amount of the active agent ranges from about 0.5 mg/mL to about 15 mg/mL, or from about 1 mg/mL to about 15 mg/mL, or from about 1 mg/mL to about 10 mg/mL; such as for example, about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL and about 10 mg/mL. On a weight/volume basis, the amount of the active agent is about 0.05 w/v % to about 1.5 w/v %, of the total weight of the topical composition.

The compositions are storage stable under standard VICH storage conditions, e.g., packaged sterile conditions at room temperature, for a period of time, e.g., 1 month or longer, 3 months or longer, 6 months or longer, or 12 months or longer.

The topical compositions of the invention include the use of a JAKi in combination with one or more pharmaceutically acceptable excipients. The excipients provide a means for solubilizing and stabilizing the JAKi compound as well as for increasing the permeability of the JAKi across and through the skin of the animal being treated with the topical composition.

The topical composition of the invention comprises a JAK inhibitor. In another aspect, the JAK inhibitor is selected from the group consisting of Formula (A) (oclacitinib; N-methyl-1-{trans-4-[methyl (7H-pyrrolo[2,3-d]pyrimidin-4-yl)amino]cyclohexyl}-methane-sulfonamide); Formula (B) (ilunocitinib; 2-(3-(4-(7H-pyrrolo[2,3-d]pyrimidin-4-yl)-1H-pyrazol-1-yl)-1-(cyclopropylsulfonyl)-azetidine-3-yl)acetonitrile); Formula (C) (1-(4-cyanotetrahydro-2H-pyran-3-yl)-3-((2-fluoro-6-methoxypyridin-4-yl)amino)-1H-pyrazole-4-carboxamide) and its stereoisomer; or Formula (D) (1-[(3R,4S)-(4-cyanotetrahydro-2H-pyran-3-yl)-3-((2-fluoro-6-methoxypyridin-4-yl)amino)-1H-pyrazole-4-carboxamide); or a pharmaceutically acceptable salt thereof. A preferred topical composition of the invention comprises oclacitinib (Formula A) or a pharmaceutically acceptable salt thereof, and in particular, oclacitinib maleate. The chemical structures of the JAK inhibitors for Formula's (A-D) are shown below:

Chemical structures:

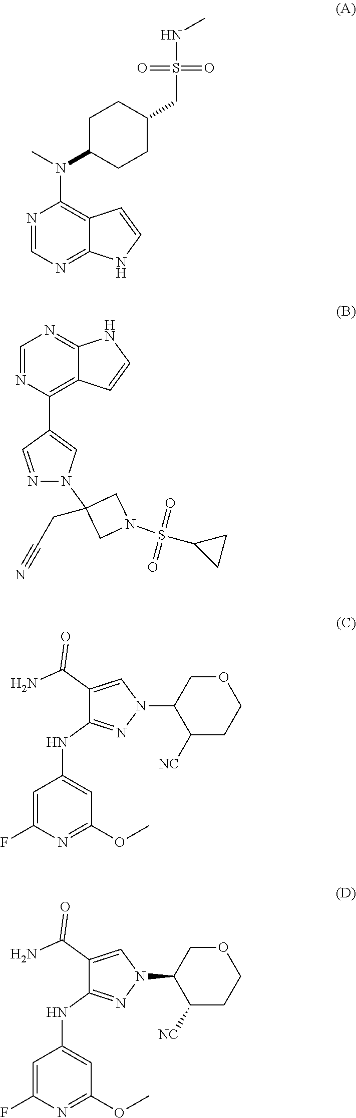

The compositions of the invention that comprise a JAK inhibitor can be formulated in a conventional manner using one or more pharmaceutically acceptable excipients which facilitate processing of the API into preparations, which can be used for topical administration. Pharmaceutically acceptable excipients are generally known to those skilled in the art and are thus included in the instant invention. Such excipients are described, for example, in “Remingtons Pharmaceutical Sciences” 19th Edition, Mack Pub. Co., New Jersey (1995).

As described above, exemplary dosages of the JAKi in the topical composition can be in the range from about 0.5 mg/mL to about 15 mg/mL; or from about 1 mg/ml to about 15 mg/mL; or from about 1 mg/mL to about 10 mg/mL. Topical compositions can comprise about 1 mg/mL, about 2.5 mg/mL, about 5 mg/mL, about 7.5 mg/ml or about 10 mg/mL. Typical administration frequencies of the composition can be administered once daily, twice daily, or as deemed necessary, depending on the size and condition of the animal.

Pharmaceutically acceptable excipients useful in the composition comprise at least one solvent or a mixture of solvents. Solvents useful in preparing the compositions of the invention can be selected from the group consisting of glycols, glycol ethers, glycerides, alcohols, dimethylsulfoxide, water, and mixtures thereof. The glycols and glycol ethers, as well as other solvents described herein can also aid in the enhancement for API skin permeation. Non-limiting examples of glycols include glycerol (glycerin), ethylene glycol, propylene glycol, butylene glycol and the low molecular weight polyethylene glycols (PEG), for example, PEG200, PEG300 and PEG400. A preferred glycol is propylene glycol or glycerol. A more preferred glycol is propylene glycol. The amount of the glycol in the composition can range from 0 w/v % to about 15 w/v %, or about 1 w/v % to about 10 w/v %, or about 2 w/v % to about 8 w/v %; of the total weight of the composition. Non-limiting examples of glycol ethers include: the mono-, di-, and tri-glycol ethers. Non-exclusive examples of the mono-glycol ethers include: ethylene glycol monomethyl ether (EGMME (EGME)), ethylene glycol monoethyl ether (EGMEE or EGEE), ethylene glycol monopropyl ether (EGMPE or EGPE), ethylene glycol monoisopropyl ether (EGMIE or EGIE), propylene glycol mono-t-butyl ether (PGMBE or PGBE), propylene glycol propyl ether (PGMPE or PGPE), propylene glycol monomethyl ether (PGMME or PGME), propylene glycol monoethyl ether (PGMEE or PGEE), and the like. Non-exclusive examples of the di-glycol ethers include: diethylene glycol monomethyl ether (DEGMME or DEGME), diethylene glycol monoethyl ether (DEGMEE or DEGEE (Transcutol®)), dipropylene glycol methyl ether (DPGMME or DPGME), diethylene glycol monobutyl ether (DEGMBE or DEGBE (butyl digol)), dipropylene glycol monomethyl ether (DPGMME or DPGME), diethylene glycol dimethyl ether (DEGDME), and the like. Non-exclusive examples of the tri-glycols include: tripropylene glycol monomethyl ether (TPGMME or TPGME), tripropylene glycol monoethyl ether (TPGMEE or TBGEE), triethylene glycol monoethyl ether (TEGMEE or TEGEE), triethylene glycol monomethyl ether (TEGMME or TEGME), and the like. The glycol ethers also include the acetylated glycol ethers, for example, diethylene monoethyl ether acetate and diethylene monobutyl ether acetate. The preferred glycol ether is selected from the group consisting of DEGMME, DEGMEE, DEGMBE and DPGMME, and mixtures thereof. A more preferred glycol ether is DPGMME, DEGMEE and DEGMME. An even more preferred glycol ether is DPGMME and DEGMEE. The most preferred glycol ether is DEGMEE. The amount of the glycol ether in the composition can range from about 10 w/v % to about 40 w/v %, or about 20 w/v % to about 35 w/v %, or about 25 w/v % to about 35 w/v %; of the total weight of the composition.

The alcohols refer to C₁-C₁₈ aliphatic alcohols and to C₄-C₆ cyclic and aromatic alcohols. The alcohols also include the fatty alcohols. Non-limiting examples of the aliphatic alcohols include ethanol, propanol, isopropanol, butanol, pentanol, hexanol, decanol, dodecanol, myristyl, cetyl, stearyl, oleic, octyldecyl, and the like. Non-limiting examples of cyclic and aromatic alcohols include cyclobutanol, cyclopentanol, cyclohexanol, benzyl alcohol, and the like. A preferred alcohol is ethanol, butanol, isopropanol and benzyl alcohol. A more preferred alcohol is ethanol. The amount of alcohol in the composition can range from about 0 w/v % to about 15 w/v % or about 0 w/v % to about 10 w/v %; or about 1 w/v % to about 10 w/v % of the total weight of the composition. The preferred alcohol, if used, is ethanol. In one aspect, the composition of the invention does not contain ethanol. In another aspect, the amount of ethanol in the composition is about 5 w/v %. In another aspect, the amount of ethanol in the composition is about 10 w/v %.

The amount of DMSO in the composition can range from about 25 w/v % to about 50 w/v %, or about 30 w/v % to about 50 w/v %, or about 30 w/v % to about 45 w/v %, or about 35 w/v % to about 45 w/v %; of the total weight of the composition. The amount of water that is added to the composition is a “qs” amount. The qs amount refers to the amount of water that is added to the composition to bring the total weight of the composition to 100%. The amount of water can range from about 10 w/v % to about 35 w/v %, or about 15 w/v % to about 30 w/v %; or about 15 w/v % to about 25 w/v % of the total weight of the composition.

Additional pharmaceutically acceptable solvents that can be used in the composition include: terpene alkaloids (for example, limonene, eucalyptol, menthol); pyrrolidones (for example, 2-pyrrolidone, N-methyl pyrrolidone and azone), glycerol formal, tetraglycol (glycofurol), tetrahydrofurfuryl alcohol, solketal, dimethyl isosorbide, citrate buffer, macrogolglycerol hydroxystearate (PEG-40 castor oil; Kolliphor EH40®) and dimethylacetamide (DMA).

In the present invention, the composition can further comprise a pharmaceutically acceptable excipient that is an anionic-, cationic- or non-ionic surfactant, or mixtures thereof. Non-limiting examples of these surfactants include: alkaline stearates (e.g., sodium, potassium, or ammonium stearate, calcium stearate, and triethanolamine stearate), alkyl sulphates (e.g., sodium laurel sulphate, sodium dodecyl sulphate, sodium cetyl sulphate), fatty acid sorbitan esters (e.g., Span® 20), polyoxyethylenated sorbitan esters (e.g., polysorbate 80), polyoxyethylenated alkyl ethers, polyethylene glycol stearate, polyoxyethylenated derivatives of castor oil (e.g., Cremaphor® EL), polyglycerol esters, caprylocaproyl macrogol-8 glyceride (Labrasol®), Kolliphor® HS15 (macrogol 15 hydroxystearate or polyoxyl 15 hydroxystearate), and the like. A preferred surfactant is caprylocaproyl macrogol-8 glyceride. The amount of surfactant in the composition can range from about 0 w/v % to about 6 w/v %, or about 0 w/v % to about 5 w/v % or about 0 w/v % to about 2 w/v % of the total weight of the composition.

In another aspect of the invention, the composition can further comprise a spreading agent (e.g., siloxane, indapole, and the like); an antioxidant (e.g., ascorbic acid, tocopherol, butylated hydroxanisole (BHA), butylated hydroxytoluene (BHT), propyl gallate, thioglycerol, citric acid and the like); a pH modifier (e.g., sodium hydroxide (NaOH), potassium hydroxide (KOH), triethanolamine, ethanolamine, hydrochloric acid (HCl), and the like). An antioxidant is preferably in the amount of about 0.1 w/v % to about 0.3 w/v % or about 0.1 w/v % to about 0.2 w/v %, or about 0.1 w/v % to about 0.15 w/v % of the total weight of the composition. The pH of the topical composition of the invention can range from about 4.5 to 7.5. A preferred pH for the topical composition ranges from about 5 to 6.5.

In another aspect, the composition of the invention can further comprise a fragrance. Non-limiting examples of fragrances include: jasmine, lavender, rose, orchid, lotus, lilac, peonia, honeysuckle, hibiscus, citrus, sandalwood, vetiver, vanilla, cinnamon, burberry and the like. In another aspect, the composition further comprises a fragrance. The amount of fragrance in the composition can be in the amount of about 0 w/v % to about 0.2 w/v % of the total weight of the composition

Non-limiting examples of topical compositions of the instant invention are shown in Table 2.

TABLE 2
Examples of Topical Compositions

| | API\hat{ } | | | Glycol | Propylene | | |
|---|---|---|---|---|---|---|---|
| | (mg/mL) | | | Ether | glycol | Labrasol | |
| Ex# | (w/v %) | DMSO (w/v %) | EtOH (w/v %) | (w/v %) | (w/v %) | (w/v %) | Water |
| 1 | 0.5-15 | 25-50 | 0-15 | 10-40 | 0-15 | 0-6 | sb |
| 2 | 0.5-15 | 25-50 | 0-10 | 10-40 | 0-15 | 0-6 | qs |
| 3 | 1-10 | 30-50 | 0-10 | 10-40 | 0-15 | 0-6 | qs |
| 4 | 1-10 | 30-50 | 0-10 | 20-35 | 1-10 | 0-6 | qs |
| 5 | 1-10 | 30-50 | 0-10 | 20-35 | 2-8 | 0-6 | qs |
| 6 | 1-10 | 30-45 | 0-10 | 20-35 | 2-8 | 0-6 | qs |
| 7 | 1-10 | 30-45 | 0-10 | 25-35 | 1-10 | 0-6 | sb |
| 8 | 1-10 | 30-45 | 0-10 | 25-35 | 2-8 | 0-6 | qs |
| 9 | 1-10 | 35-45 | 0-10 | 20-35 | 0-15 | 0-6 | qs |
| 10 | 1-10 | 35-45 | 0-10 | 20-35 | 1-10 | 0-6 | qs |
| 11 | 1-10 | 35-45 | 0-10 | 20-35 | 2-8 | 0-6 | qs |
| 12 | 1-10 | 35-45 | 0-10 | 25-35 | 1-10 | 0-6 | sb |
| 13 | 1-10 | 35-45 | 0-10 | 25-35 | 2-8 | 0-6 | sb |
| 14 | 1-5 | 30-45 | 0-10 | 20-35 | 1-10 | 0 | sb |
| 15 | 1-5 | 35-45 | 0-10 | 20-35 | 1-10 | 0 | qs |
| 16 | 1-5 | 35-45 | 0 | 20-35 | 1-10 | 0 | qs |
| 17 | 1-5 | 35-45 | 5 | 20-35 | 1-10 | 0 | sb |
| 18 | 1-5 | 35-45 | 10 | 20-35 | 1-10 | 0 | qs |
| 19 | 1-5 | 35-45 | 0 | 25-35 | 1-10 | 0 | sb |
| 20 | 1-5 | 35-45 | 0-10 | 25-35 | 2-8 | 0 | sb |
| 21 | 1-5 | 35-45 | 0 | 25-35 | 2-8 | 0 | qs |
| 22 | 1-5 | 40 | 0 | 30 | 5 | 0 | qs |
| 23 | 1-5 | 35 | 1-5 | 30 | 5 | 0 | qs |
| 24 | 1-5 | 40 | 1-5 | 30 | 5 | 0 | qs |
| 25 | 1-5 | 40 | 0 | 25-35 | 2-8 | 0 | qs |

\hat{ }Concentration of oclacitinib. Each composition optionally includes an antioxidant (0.1-0.3 w/v %); and is pH adjusted as needed. The qs amount of water ranges from about 10-35 w/v %.

In another aspect, the topical composition of the invention can be administered alone or concomitantly with other pharmaceutical agents, whether administered orally, parenterally or topically, that may be administered to the animal to aid in the healing of the dermatological disorders or other disorders. For example, other known pharmaceutical agents include, for example, antibiotics (e.g., neosporin, fusidic acid, amikacin, thiostrepton, metronidazole, clindamycin, cephalosporins, fluoroquinolones, tetracyclines and the like); anti-fungals (e.g., ketoconazole, sulconazole, miconazole, amphotericin, fluconazole, nystatin and the like); anti-inflammatories (e.g., hydrocortisone, diamino-diphenyl sulphone, triamcinolone acetonide, menthol, non-steroidal anti-inflammatory drugs (NSAIDS (e.g., meloxicam, flunixin meglumine, carprofen, aspirin, ketoprofen, naproxen, diclofenac and the like), and the like); anti-septics (e.g., chlorhexidine, povidone-iodine, chloroxylenol, hexachlorophene, isopropyl alcohol, benzalkonium chloride, hydrogen peroxide and the like); steroids (e.g., clobetasol proprionate, flurandrenolide, betamethasone diproprionate, diflorasone diacetate, desoxymethasone, halobetasol proprionate, fluocinonide, halcinonide, flurandrenolide, dexamethasone, betamethasone valerate and the like); analgesics (e.g., capsaicin, camphor, menthol, gingerol, lidocaine, bupivacaine, NSAIDS, ibuprofen and the like)

The topical compositions of the invention, as described herein, can be manufactured by common means for preparing a solution. First, the solvents, dimethylsulfoxide, ethanol, glycol ether (DEGMEE, DEGMME or DEGMBE), propylene glycol and optionally Labrasol® are dispensed and mixed together. The antioxidant (e.g., BHA or BHT) and the JAKi are then added sequentially to the solvent mixture and mixed until dissolved. Alternatively, the active agent can be dissolved in DMSO prior to mixing with the other solvents and then, optionally, adding the surfactant and antioxidant. The mixture is pH adjusted to about pH 5.4, as needed. The final weight of the composition is adjusted (qs) with water to 100%. The final solution is filtered and filled into spray type bottles. Each bottle can contain about 30-240 mL of the composition of the invention. For example, bottles can contain about 30 mL, 45 mL, 60 mL, 90 mL, 100 mL, 120 mL, 150 mL, 175 mL, 190 mL, 200 ml, 225 mL or 240 mL of the composition of the invention. Bottles cans also contain volumes between the 30-240 mL, for example 40 mL, 80 mL, 140 mL 170 mL, and the like.

Oclacitinib (7.5 mg/mL) stability at 1 month accelerated stability (40° C./75% relative humidity) ranged from about 97.2 to 99.3% for compositions comprising 0.15-0.2 w/v % BHT.

1. A topical solution composition comprising a) a JAK inhibitor selected from the group consisting of Compound A, Compound B, Compound C and Compound D; or a pharmaceutically acceptable salt thereof; 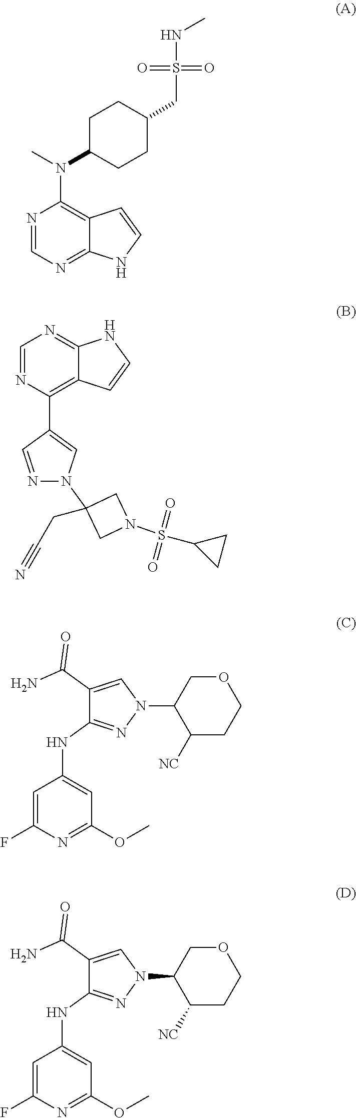
b) a pharmaceutically acceptable solvent mixture comprising a glycol, glycol ether, dimethylsulfoxide and water; and c) optionally, an antioxidant and pH modifier.

2. The composition of claim 1, wherein the JAK inhibitor is Compound A, or a pharmaceutically acceptable salt thereof; and the glycol is propylene glycol and the glycol ether is DEGMEE, DPGMME or DEGMBE.

3. The composition of claim 2, wherein the glycol ether is DEGMEE.

4. The composition of claim 3, wherein the composition further comprises an antioxidant and a pH modifier.

5. The composition of claim 4, wherein the antioxidant is BHA, BHT, or a mixture thereof; and the pH modifier is NaOH or KOH.

6. The composition of claim 1, wherein the glycol is in the amount of about 2-8 w/v % of the total weight of the composition; the glycol ether is in the amount of about 25 w/v % to about 35 w/v % of the total weight of the composition; the dimethylsulfoxide is in the amount of about 30 w/v % to about 45 w/v % of the total weight of the composition.

7. The composition of claim 6, wherein the JAK inhibitor is in the amount of about 1 mg/mL to about 10 mg/mL.

8. The composition of claim 2, wherein the pharmaceutically acceptable salt is the maleate salt.

9. The composition of claim 1, further comprising ethanol in the amount of about 1 w/v % to about 5 w/v % of the total weight of the composition.

10. The topical solution composition of claim 1, comprising a Formula (A) compound, or pharmaceutically acceptable salt thereof, in the amount of about 1 mg/mL to about 5 mg/mL, the glycol, propylene glycol, in the amount of about 2 w/V % to about 8 w/v %, the glycol ether that is DEGMEE in the amount of about 20 w/v % to about 35 w/v %, dimethylsulfoxide in the amount of about 30 w/v % to about 45 w/v % and wherein the w/v % is a percentage of the total weight of the composition.

11. The composition of claim 10, further comprising an antioxidant and pH modifier.

12. The composition of claim 11, wherein the antioxidant is BHA, BHT, or a mixture thereof.

13. The composition of claim 12, wherein the propylene glycol is in the amount of about 5 w/v %, DEGMEE is in the amount of about 30 w/v %, dimethylsulfoxide is in the amount of about 40 w/v %, and the antioxidant is BHT; and wherein the pH of the composition ranges from about 4.8 to 6.0.

14. Use of the topical solution composition of claim 1, for the treatment and prevention of a dermatological disorder selected from allergic dermatitis, atopic dermatitis, hot spot, pruritis, and symptoms thereof, in a non-human animal in need thereof.

15. A method of treating a dermatological disorder selected from allergic dermatitis, atopic dermatitis, hot spot, pruritis, and symptoms thereof, in a non-human animal by administering the composition of claim 1.

16. A method of treating a dermatological disorder selected from allergic dermatitis, atopic dermatitis, hot spot, pruritis, and symptoms thereof, in a nonhuman animal by administering the composition of claim 13.

17. The method of claim 15, wherein the non-human animal is canine or feline.

18. The method of claim 16, wherein the non-human animal is canine or feline.
